(19) United States
(12) Patent Application Publication
(10) Pub. No.: US 2022/0128559 A1
(43) Pub. Date: Apr. 28, 2022

(54) Surveillance of SARS-CoV-2 Through Mask Testing

(71) Applicant: VMRD, Inc., Pullman, WA (US)

(72) Inventors: Dennis Scott Adams, Pullman, WA (US); Ethan Jackson Adams, Colfax, WA (US); Siddra Arielle Hines, Pullman, WA (US); Andrew Kenneth Johnson, Moscow, ID (US); Donald Patrick Knowles, JR., Pullman, WA (US); Sampathkumar Srikanth, Pullman, WA (US); Joanna Rzepka, Pullman, WA (US)

(73) Assignee: VMRD, Inc., Pullman, WA (US)

(21) Appl. No.: 17/507,604

(22) Filed: Oct. 21, 2021

Related U.S. Application Data
(60) Provisional application No. 63/104,176, filed on Oct. 22, 2020.

(51) Int. Cl. G01N 33/569; G01N 1/28; G01N 1/40

(52) U.S. Cl. CPC G01N 33/56983; G01N 1/286; G01N 2800/26; G01N 2001/2873; G01N 1/4044

(57) ABSTRACT

Representative implementations of devices and techniques provide an exemplary system and method for detection and surveillance of an infectious agent, and particularly an agent that is spread through respiratory aerosols. In an embodiment, the system and method include retrieving a face covering that contains oral and/or nasal, and/or skin secretions and/or skin flora on or within a material of the face covering, removing one or more samples of the material of the face covering that contains the secretions from the face covering, and depositing the one or more samples into a predetermined formulation.

### PRIORITY CLAIM AND CROSS-REFERENCE TO RELATED APPLICATION

This application claims the benefit under 35 U.S.C. § 119(e)(1) of U.S. Provisional Application No. 63/104,176, filed Oct. 22, 2020, which is hereby incorporated by reference in its entirety.

### BACKGROUND

A primary challenge in preventing the transmission of infectious agents, such as SARS-CoV-2 and other organisms, is the lack of convenient, non-invasive continual surveillance for the presence of viral and other infectious organisms, especially in asymptomatic carriers. Typical detection methods include the use of nasal swabs, which can be prone to false negative results. For example, since it can take roughly 5 to 7 days after contracting a virus for a sufficient population to grow in the nasal sinus, the virus may not be present in a great enough concentration at the time of collection for detection. A false negative can result, allowing the carrier to unknowingly spread the infectious agent to others. This can be problematic in the workplace, at schools, and in other situations where regular interaction with others, often in proximity, is commonplace.

Further, there can be a delay associated with nasal swab testing (and like testing methods) and receiving the associated test results, where the delay also gives time for unknowing transmission of the virus/organism. The need to schedule the test with a clinic can be one source of delay, particularly if the available clinic is overwhelmed. Of course, the workload of clinics and labs is compounded when during a pandemic or other serious outbreak.

### SUMMARY

SARS-CoV-2 is transmissible for several days prior to symptoms, as well as by asymptomatic individuals, posing significant risk for spread within populations. The use of facemasks has been encouraged by public health authorities to slow viral transmission. As part of biosecurity efforts, facemasks can also be tested for the presence of SARS-CoV-2. Facemask testing is a practical tool for ongoing surveillance to reduce potential SARS-CoV-2 burden within facilities and is more feasible on a large scale than nasopharyngeal/nasal swabs. As the oral and nasal cavities are the primary sites for viral shedding, facemasks that cover these areas provide a concentrated sample of accumulated droplets, aerosols, and viral particles trapped by the mask. This sample also represents the full duration of potential shedding time for which the mask was worn, rather than the single point of a swab collection.

On-site mask sample preparation by the mask wearer (folding and sampling) provides samples to be tested by PCR for workplace surveillance. Briefly, individuals collect samples from their own surgical, N95, KN95, or other masks according to a designated sampling procedure using individually assigned punch tools and then place samples in transport media. This media stabilizes the nucleic acids and inactivates SARS-CoV-2 (and other pathogens), allowing safe transport to a laboratory setting for PCR testing.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

For this discussion, the devices and systems illustrated in the figures are shown as having a multiplicity of components. Various implementations of devices and/or systems, as described herein, may include fewer components and remain within the scope of the disclosure. Alternately, other implementations of devices and/or systems may include alternate or additional components, or various combinations of the described components, and remain within the scope of the disclosure. Shapes and/or dimensions shown in the illustrations of the figures are for example, and other shapes and or dimensions may be used and remain within the scope of the disclosure, unless specified otherwise.

FIG. 1 illustrates an example mask with samples of the mask removed and a collection container for receiving the samples, according to an embodiment.

FIG. 2 illustrates an example technique for collecting samples from a mask and a collection container for receiving the samples, according to an embodiment.

FIG. 3 illustrates an example mask worn on a person.

FIG. 4 illustrates an example kit for collecting samples from a mask, including a hole punch, a tweezer, a collection container including a formulation for receiving the samples, and a stand for the collection container, according to an embodiment.

FIG. 5 illustrates an example technique for collecting samples from a mask using a hole punch, according to an embodiment.

FIG. 6 illustrates an example folded mask with samples removed, according to an embodiment.

FIG. 7 illustrates an example unfolded mask with samples removed, according to an embodiment.

FIG. 8 illustrates an example technique for collecting samples from a mask into a collection container for receiving the samples, according to an embodiment.

FIG. 9 illustrates an example unfolded mask with a removable insert, according to an embodiment.

FIG. 10 illustrates an example unfolded mask with a removable insert, according to another embodiment.

FIG. 11 illustrates an example technique for collecting samples from a mask insert using a hole punch, according to an embodiment.

FIG. 12 illustrates an example folded insert with samples removed, according to an embodiment.

FIG. 13 shows example formulation components and concentrations, according to an embodiment.

FIG. 14 shows an example flow chart for a process of using a mask for detection of SARS-CoV-2 and other infectious agents, according to an embodiment.

### DETAILED DESCRIPTION

### Overview

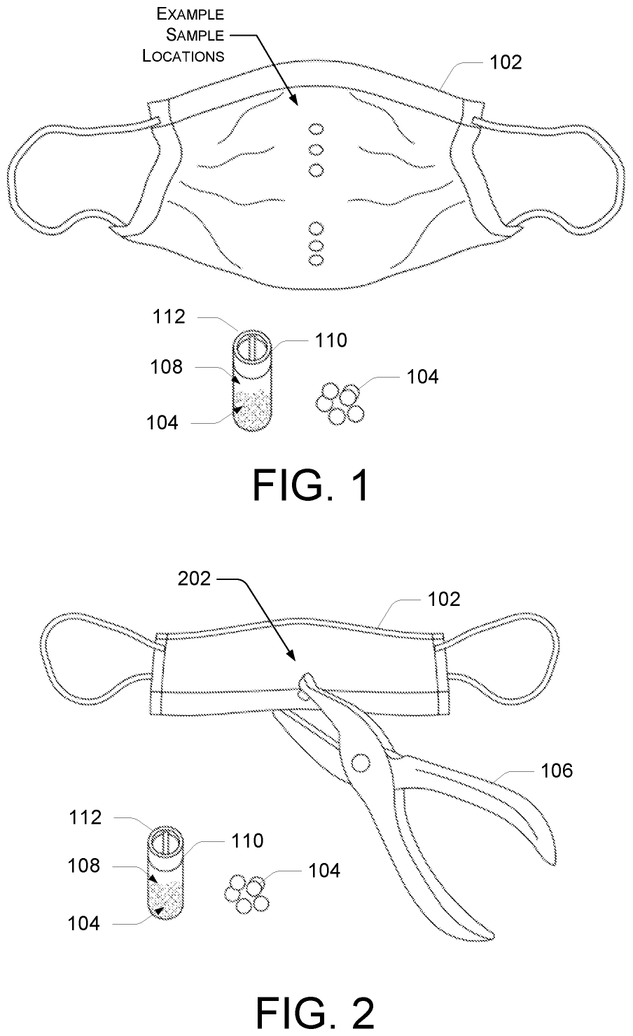

Referring to FIGS. 1 and 2, representative implementations of methods, systems, and techniques provide simple and convenient surveillance of infectious agents through testing of face coverings 102 (i.e., masks and other face coverings comprising natural or man-made materials). Members of a group can provide and prepare samples 104 from their face coverings 102 in an on-site setting and at regular intervals if desired. The samples 104 can be transported for lab-testing to determine the presence of a pathogen, for instance.

Briefly, individuals collect samples 104 from their own surgical, N95, KN95, or other masks 102 according to a designated sampling procedure using individually assigned punch tools 106 and then place the samples 104 in a transport media 108. The media 108 stabilizes the nucleic acids and inactivates SARS-CoV-2 (and other pathogens) in the samples 104, allowing safe transport to a laboratory setting for real-time PCR testing, for example. The disclosed technique can be effective for the detection and surveillance of SARS-CoV-2, as well as other important respiratory pathogens such as influenza A and B, respiratory syncytial virus (RSV), methicillin-resistant Staphylococcus aureus, and others.

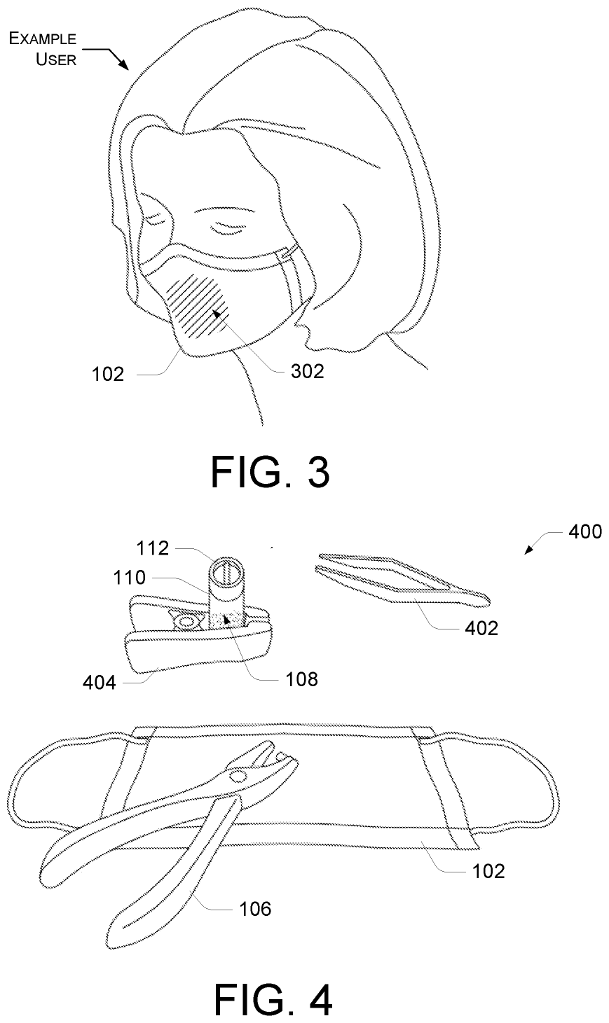

In various implementations, as shown at FIG. 3, a person wears a face covering (hereinafter “mask 102”) throughout at least a part of the day, depositing one or more of oral secretions, nasal secretions, skin secretions, and skin flora (hereinafter “secretions 302”) into the mask 102. The cumulative secretions 302 are collected in the mask 102 during the time it is worn. This results in a high concentration of secretions 302, including infectious agents if present, on the inside surface and/or within the material of the mask 102. In an implementation, the user wears the mask 102 for more than 1 minute (to collect sufficient secretions 302). Alternately, a user may wear the mask 102 for 1 minute or less and still collect sufficient secretions 302 for detection.

At the end of the period, the mask 102 is sampled (e.g., by the user) and the samples 104 are collected in a container 110 of transport media 108 having a specific formulation for eluting, preserving and storing the mask samples 104 at room temperature. The samples 104 can then be tested for the infectious agent through molecular and/or antigen testing.

The convenience of the methods, systems, and techniques disclosed herein mean that a person can collect samples 104 from their own mask 102, rather than needing to schedule an appointment at a clinic. This also suggests that the quality of the test samples is better (a higher concentration of secretions 302) without the risk of exposing a clinician to a virus, as compared to nasal swab testing. The non-invasive nature of the techniques also improves the likelihood of user-compliance for more reliable tracking.

The user can deposit the mask samples 104 in a collection vessel 110 containing the formulation 108, and then use any convenient means to get the container 110 of samples 104 to a lab for molecular or antigen testing. The formulation 108 preserves the collected samples 104 in a stable non-infectious condition at room temperature for an extended time (over one month). Thus, the collection containers 110 could be put in the mail, dropped into a drop-box, or otherwise conveniently transported to a lab for testing. No special handling or refrigeration is needed during transport, which makes the process 1400 more robust and much less expensive than if refrigeration or other special handling was required. In an implementation, the container 110 includes 1.0 ml of transport media 108 or less. Alternately, the container 110 may include more than 1.0 ml of transport media.

Since the mask samples 104 are sent to the lab for testing instead of a person having to report to the lab to be tested, the delays associated with lab scheduling for testing can be reduced significantly. Also, additional labs can be available for testing the collected samples 104 than are available for processing individuals, further increasing throughput and reducing test delays. In general, test results of the collected samples 104 can be available within 2 hours (or one-half hour in some molecular tests), providing quick and accurate results that can be acted on swiftly if necessary to isolate an infected person.

Further, since mask samples 104 are sent to the lab for testing instead of sending the mask 102 itself to the lab for testing, the burden on the lab is manageable. For example, no laboratory could economically process hundreds or thousands of masks 102 in a relatively short amount of time. Each mask 102 would have to be processed with the technician using new PPE and in a biological safety hood. This is because multiple human pathogens (SARS-CoV-2, cold viruses, flu, etc.) may be present. In addition to requiring new PPE (masks & gloves) between masks 102, the time to sample and process each mask 102 is not economically feasible. Many fewer masks 102 (in orders of magnitude) could be tested than is possible using the mask samples 104 as disclosed herein. The samples 104 can be taken to any lab, anytime, without creating an undue burden on the lab. The short turn-around time allows for reliable surveillance and tracking of an individual or a group, with enough time to take action (e.g., quarantine, medical treatment, etc.) when necessary.

Tracking and trending of larger groups is made more affordable and convenient. To save expense, some pooling of collected samples 104 can be helpful and can also make more efficient use of test reagents, lab technicians, and test machines. A department, a workplace organization, or a class at a school, for example, can pool the samples 104 for groups of members of the department, organization, or class in a common test batch. The collected samples 104 can be batch tested by group, on a convenient cycle. Any groups that show positive results can be further tested to determine the individuals within the group that are carrying the infectious agent.

This process can be highly effective in preventing agent transmission within the workplace, school, and the like. Because it merely relies on each of the members wearing a mask 102 at least a part of the day, rather than scheduling periodic nasal swab tests (or the like), there is no disrupting the continuity of the business or group. The less invasive testing is also easier on children and sensitive individuals. The members of the group or of identified sub-groups can be part of regular on-going mask 102 tests that track and trend the agent within the group or sub-groups. Since the agent can be detected quickly (less than 2 hours at the lab), the infected person(s) can be isolated sooner, preventing further spread of the agent to others.

Various implementations and arrangements are discussed with reference to viral infectious agents (such as SARS-CoV-2) and face coverings such as masks 102. While specific examples are mentioned, this is not intended to be limiting, and is for ease of discussion and illustrative convenience. The methods, systems, and techniques discussed are applicable to any type of infectious agents, including viral and bacterial, as well as any other organism, flora, element, compound, nucleic acid or other substance desired to be detected, that can be associated with, combined with, or carried on human or animal nasal, oral, or skin secretions 302, respiratory or perspiration droplets and aerosols, live or dead skin cells, and the like. Each of these are generically referred to herein as an “agent.”

Further, the term “mask 102” is used herein to refer to any face mask, clinical mask, dust mask, face covering, respirator, handkerchief, item of clothing, cloth, accessory, and the like that can be disposed over the mouth and/or nose of a human or animal and arranged to collect or retain some portion of the respirations or perspiration, the oral, nasal, and/or skin secretions, and/or the skin flora, and the like (collectively, secretions 302), of the human or animal.

Implementations are explained in more detail below using a plurality of examples. Although various implementations and examples are discussed here and below, further implementations and examples may be possible by combining the features and elements of individual implementations and examples.

### EXAMPLE PROCESSES AND IMPLEMENTATIONS

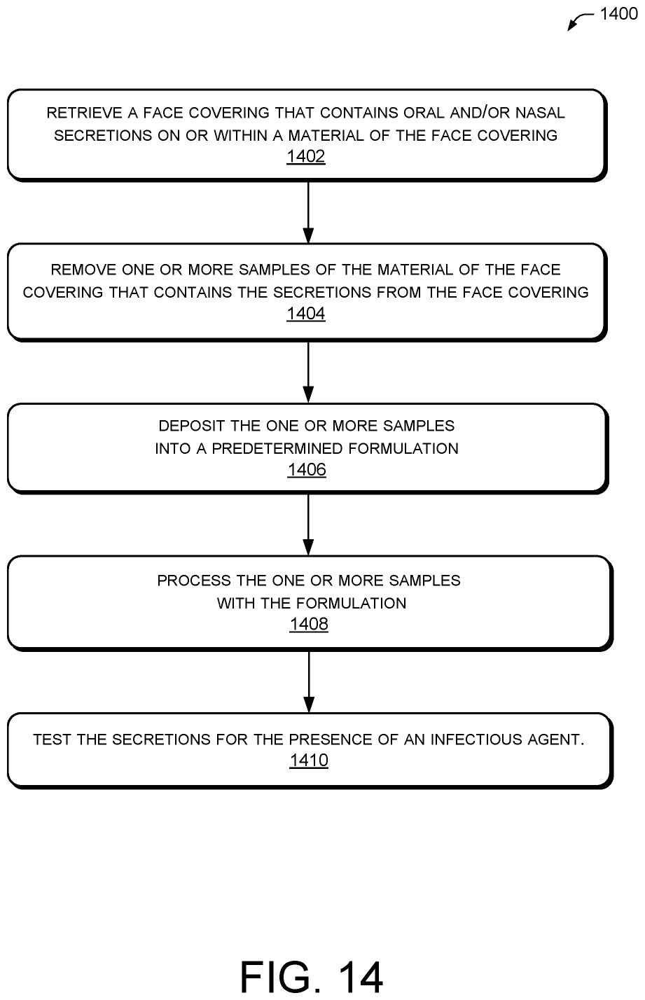

FIGS. 3-12 and 14 (and also referring to FIGS. 1 and 2) illustrate example processes 1400 of surveillance of infectious agents and the like through mask 102 testing (as discussed above), according to various embodiments, including eluting, preserving, and storing mask samples 104 at room temperature. The processes 1400 described are examples that are not intended to be limiting. Further, the processes 1400 described with respect to FIGS. 1-12 and 14 may also describe a corresponding apparatus, structure, system, or the like. For example, one embodiment of a system “kit” 400 for surveillance of infectious agents and the like through “mask 102” testing is shown at FIG. 4.

The order in which the processes 1400 are shown and described is not intended to be construed as a limitation, and any number of the described process steps can be combined in any order to implement the processes 1400, or alternate processes. Additionally, individual steps may be added or deleted from the processes 1400 without departing from the spirit and scope of the subject matter described herein. Furthermore, the processes 1400 can be implemented in any suitable materials, or combinations thereof, without departing from the scope of the subject matter described herein. In alternate implementations, other techniques may be included in the processes 1400 in various combinations, and remain within the scope of the disclosure.

Referring to FIG. 3, a person wears a mask 102 (as defined above) for a certain duration. For instance, the person can wear the mask 102 for 4 to 8 hours, or for a similar duration that the person is at work, at school, or the like. During the time the mask 102 is worn, the person secretes oral, nasal, and/or skin secretions, and/or skin flora (i.e., secretions 302) into the mask 102, which collect on the inside surface of the mask 102 and/or within the material of the mask 102. In an alternate embodiment, an animal wears a mask 102 (as described above) for a certain duration, with like collection of secretions 302 from the animal's respiration or shed skin cells.

The mask 102 may be comprised of one or more of paper, textile, polymer, or any man-made or naturally occurring material, including N95 and KN95 masks and materials. The mask 102 may be more or less absorbent in material and construction. Since samples 104 are to be cut from the mask 102, the ease of cutting the mask 102 can be a consideration as to its composition.

While the person wears the mask 102 (regardless of the presence of detectable symptoms), the secretions 302 collect in a cumulative mariner on or in the material of the mask 102 becoming more concentrated over the duration of wear. The greater the duration of wear, the higher the concentration of secretions 302 (and the agent if present) are possible. In other words, if an agent is present in the secretions 302, the collected secretions 302 likely present a higher concentration of the agent than is generally found during a nasal swab test, or the like, making detection of the agent more reliable. This can help to detect an agent carrier earlier, particularly when the carrier is asymptomatic. It can also help to track the movement of the agent through a group or community earlier and with the ability to respond to the movement sooner.

Referring to FIG. 4, a “surveillance kit” 400 can be made available to the person for their own use, or it can be administered by another. An example kit 400 may include a mask 102, such as a disposable clinical mask, or any other type of mask that is well suited to the surveillance process and/or is inexpensive. A mask 102 that is absorbent and that is easy to cut samples 104 from is well suited to the process.

An example kit 400 can include a cutting device 106 for taking samples 104 from the mask 102, such as a hole punch as shown. Other devices may also be used, such as blades, scissors, punches, and so forth. Ideally, the cutting device 106 should not be used to cut samples 104 from multiple masks 102 worn by different people, unless the device is sufficiently cleaned between masks 102 or the samples 104 are to be aggregated into a group pool. Further, when tracking is desired, it is helpful for the user to clean the cutting device 106 after each mask 102 sampled by the device 106 (even when worn by the same person).

An example kit 400 may include a tweezer 402 or like device for removing the samples 104 from the mask 102 or for handling the samples 104 once they are removed from the mask 102. Appropriate cleaning protocols, such as wiping with 70% alcohol for the tweezer 402 are like those used for the hole punch (or cutting device 106). Further, the surface used for cutting and handling the samples 104 should also be cleaned between masks 102 under the same conditions mentioned above, to avoid cross-contamination.

An example kit 400 includes a container 110 for collecting the samples 104. The container 110 need only be large enough to hold a reasonable quantity of samples 104 (for instance about six to ten cut-out mask samples 104 the size of hole punch cut-outs). The container 110 should only be used to collect the samples 104 from a single mask 102, unless aggregation is desired (in which case a larger container 110 may be used). The container 110 is sealable (with a cap 112, for instance) to prevent contamination of the samples 104, and to ensure the preservation of the samples 104 during transportation and storage.

The container 110 includes a formulation 108, which is discussed further below. The samples 104 are immersed into or coated with the formulation 108 when deposited into the collection container 110.

As shown at FIG. 4, the kit 400 may also include a stand 404 for the container 110, to make it easier to drop the samples 104 into the container 110 without cross-contamination of the samples 104 or exposing the test administrator (if it is another person) to the agent. The container stand 404 may be any simple or complex device (like a clip, for example) that holds the container 110 (or multiple containers) upright.

In various implementations, a surveillance kit 400 may include additional items as desired, or alternate items to perform the equivalent tasks described herein. A person having skill in the art will recognize alternate items or alternate forms of the items described that are within the scope of the disclosure.

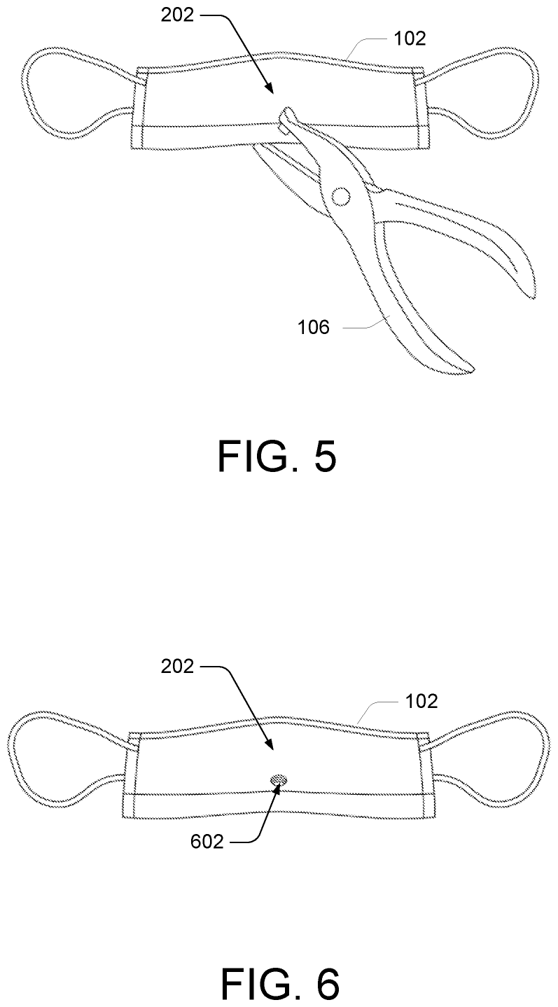

Referring to FIG. 5, the mask 102 can be folded prior to sampling to make the sampling process more efficient. The mask 102 may be folded once, twice, or more times so that a single punch (or cut) results in multiple samples 104. FIG. 6 shows a folded mask 102 with a single sample punch 602 made, where the single punch 602 delivers multiple mask samples 104 (as shown by the multiple mask layers in the punch hole 602). Note that the samples 104 may be taken from a central region of the mask 102 (in the vicinity of the central vertical axis of the mask 102 as worn) to ensure that a high-secretion concentration sample 104 is taken. Several sample punches 602 may be taken if desired, or a single punch 602 through multiple mask 102 folds can be sufficient.

Additionally, folding the mask 102 prior to cutting the samples 104 from the mask 102 can minimize or eliminate contamination by having the punch tool 106 surface only touch the outside layer of the folded mask 102 (on both sides of the mask 102). The user can fold the mask 102 such that the outer layers of folds of the folded mask 102 comprise the edges of the mask 102, which can have a lower concentration or no concentration of secretions 302.

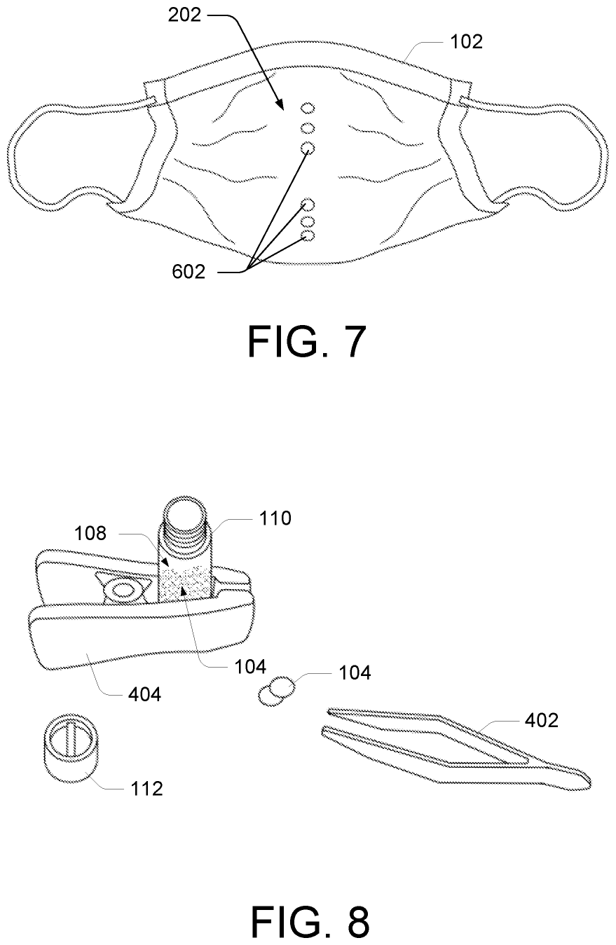

FIG. 7 shows the sampled mask 102 unfolded, after having been folded a number of times prior to punching the samples 104. As can be seen, multiple samples 104 are removed from the mask 102 in a high-secretion area 202 of the mask (e.g., the central region of the mask 102). This can be the result of multiple punches 602 or the result of a single punch 602 of a multiple-folded mask 102.

Referring to FIG. 8, the cut-out mask samples 104 are placed into the collection container 110 with the formulation 108. The samples 104 may be placed into the container 110 using the tweezers 402, or with another device that isolates the samples 104 from direct handling by a person. This prevents cross-contamination of the samples 104 as well as preventing another person administering the test from being potentially exposed to the agent (if present). The cap 112 is placed on the container 110 to seal the container 110. The container 110 may be marked with identifying information if desired along with the date and time, as well as any other pertinent information, prior to delivering the container 110 to a lab for testing.

The formulation 108 within the container 110 coats or is absorbed by the mask samples 104 placed inside the container 110. In some examples, the formulation 108 has the benefits of eluting the biological matter (and particularly the agent) of the secretions 302 from the mask 102 material of the samples 104. In the examples, the formulation 108 can act as a solvent, pulling the biological matter from the material of the mask 102, without degrading the material of the mask 102 to a substance that is difficult to separate from the biological matter. In an embodiment, the mask 102 material is not dissolved by the formulation 108, making the material of the mask 102 easily separated from the agent and biological matter. In some alternate implementations, it may not be necessary to separate the agent from the mask 102 material to reliably detect the presence of the agent in the mask sample 104. In the alternate implementations, the formulation 108 may or may not elute the biological matter from the mask 102 material.

Additionally, the enzymes (e.g., Ribonucleases (RNAses)) of the nasal and oral secretions 302 may be inactivated by the formulation 108, preserving the viral genome components and allowing the agent of interest to be separated from the remainder of the biological material (via nucleic acid extraction, for example). Once the agent is isolated, molecular or antigen testing, or the like, may be used to detect or identify the agent and to determine its concentration or population in the samples 104. For example, the nucleic acid (i.e., genetic material) of the agent can be detected and identified or studied using various molecular testing techniques at the lab, including polymerase chain reaction (PCR) techniques, where a small sample of the agent is rapidly copied to amplify the size of the sample, or the like. Alternately, an antigen test may be performed to detect specific proteins on the surface of the agent molecules. In various embodiments, other forms of tests (known or not yet discovered) may be used to identify the presence and concentration of an agent in the mask samples 104.

The formulation 108 has the additional benefits of preserving the nucleic acid of the agent for transportation and storage at room temperature. In an embodiment, the formulation 108 is capable of preserving the nucleic acid for more than one month at room temperature, with no loss in test accuracy. In an embodiment, the formulation 108 also inactivates the agent (e.g., SARS-CoV-2 and other pathogens) as part of the preservation.

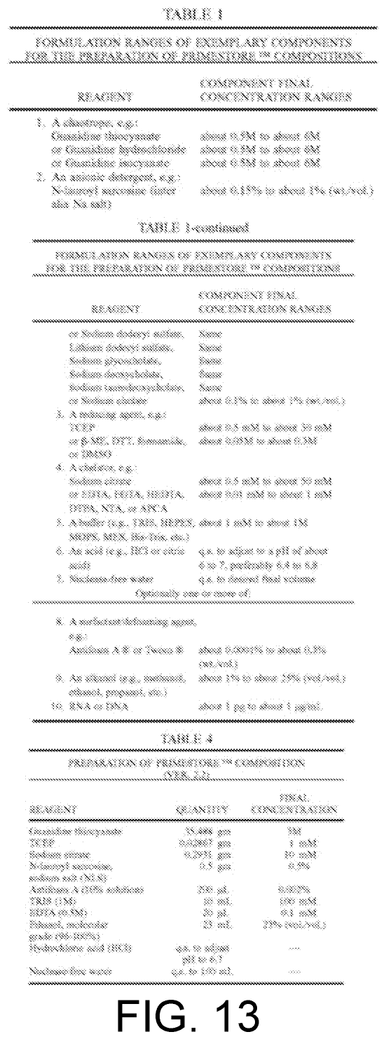

In various implementations, the formulation 108 comprises a chaotrope, alcohol, and ionic detergent. For example, in one implementation, the formulation 108 may comprise a PrimeStore™ composition, as described in U.S. Pat. No. 9,683,256 to Fischer et al. (see FIG. 13). In various implementations, the formulation 108 solution may include additional active or inactive components. Examples of chaotropes include but are not limited to: guanidine hydrochloride, guanidine thiocyante, potassium thiocyante, and urea. In an example preferred implementation of the formulation 108, the chaotrope comprises guanidine thiocyanate at 20-30%. Examples of alcohols that may be used include but are not limited to: isopropyl alcohol, methanol, and ethanol In an example preferred implementation of the formulation 108, the alcohol comprises ethanol at 19-25%. Examples of ionic detergents that may be used include but are not limited to: ammonium lauryl sulfate, sodium lauryl sulfate, and N-lauroylsarcosine sodium salt. In an example preferred implementation of the formulation 108, the ionic detergent comprises N-lauroylsarcosine sodium salt at <0.7%. Additional formulations 108 include those containing detergent with or without alcohol and chaotrope. The remaining fractions of the formulation 108 may include one or more of various inert ingredients to facilitate the use of the formulation 108 with the mask samples 104, and for ease in dispensing the formulation 108 into the containers 110.

Additional features and benefits of the formulation 108 and the techniques for surveillance of agents through mask 102 testing will be apparent to persons having skill in the art, as well as to clinicians familiar with the treatment of infectious diseases, and those familiar with the desirability of such surveillance.

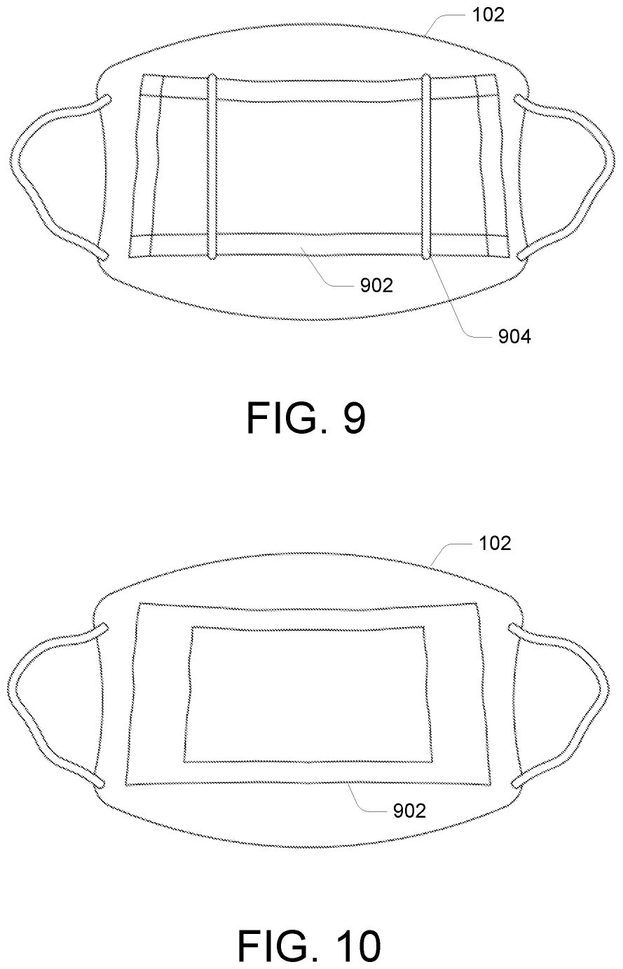

The process 1400 disclosed above may also be applied to other forms of face coverings (e.g., kerchiefs, bandanas, neck gaiters, scarves, balaclavas, and so forth) or other items worn over the face of the user. For example, referring to FIGS. 9-12, in various implementations, an insert 902 may be added to a mask 102 (or the like) to provide a surface or a material for collecting secretions 302. In the implementations, the insert 902 may be reusable, disposable, or the like, and may be used with a mask 102 when the user does not desire to destroy the mask 102 by punching samples 104 from the mask 102. In that case, the user can collect the samples from the insert 902 instead.

Referring to FIGS. 9 and 10, one or more inserts 902 can be removably coupled to the inside surface of a mask 102 (or the like) using removable couplers (such as hook and loop fasteners, snaps, buttons, etc.), using straps 904, using pockets or sleeves in the mask 102, and various other techniques. An insert 902 can also be added to the inside surface of a face shield or any other device that is worn in front of a user's face and in close proximity (e.g., within 12″) of the user's face. A fresh insert can be added to the mask 102 at the beginning of a predetermined duration, which can collect the user's secretions throughout the duration.

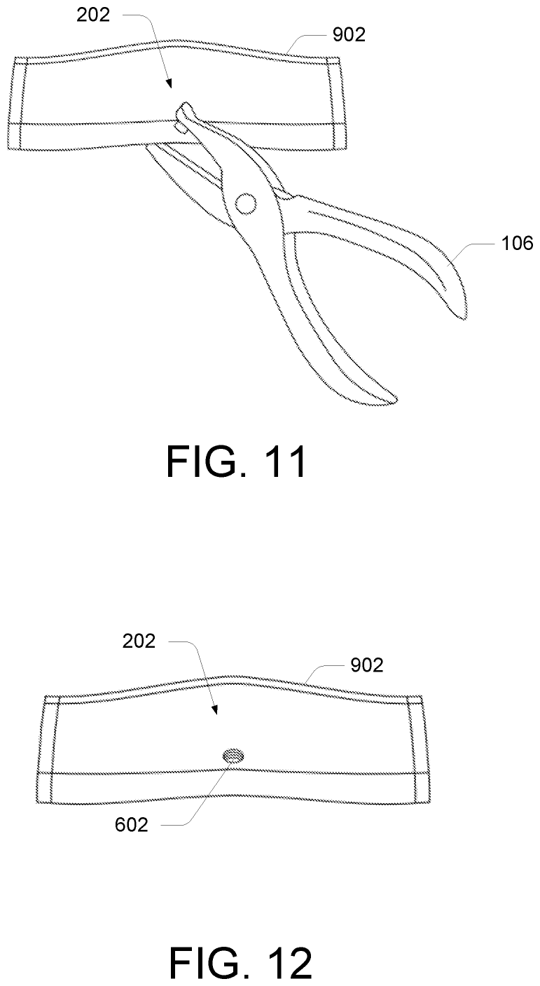

Referring to FIGS. 11 and 12, the insert 902 is removed from the mask 102. The insert 902 can be folded prior to sampling to make the sampling process more efficient. The insert 902 may be folded once, twice, or more times so that a single punch 602 (or cut) results in multiple samples 104. FIG. 12 shows a folded insert 902 with a single sample punch 602 made, where the single punch 602 delivers multiple samples 104 (as shown by the multiple insert layers in the punch hole 602). Note that the samples 104 may be taken from a central region of the insert 902 (in the vicinity of the central vertical axis of the insert 902 as worn) to ensure that a high-secretion concentration sample 104 is taken. Several sample punches 602 may be taken if desired, or a single punch 602 through multiple insert 902 folds can be sufficient.

Additionally, folding the insert 902 prior to cutting the samples 104 from the mask 102 can minimize or eliminate contamination by having the punch tool 106 surface only touch the outside layer of the folded insert 902 (on both sides of the insert 902). The user can fold the insert 902 such that the outer layers of folds of the insert 902 comprise the edges of the insert 902, which can have a lower concentration or no concentration of secretions 302.

Multiple samples 104 are removed from the insert 902 in a high-secretion area 202 of the insert 902 (e.g., the central region of the insert 902). This can be the result of multiple punches 602 or the result of a single punch 602 of a multiple-folded insert 902.

The cut-out samples 104 are placed into the collection container 110 with the formulation 108. The formulation 108 within the container 110 coats or is absorbed by the insert samples 104 placed inside. The samples 104 may be placed into the container 110 using the tweezers 402, or with another device that isolates the samples 104 from direct handling by a person. This prevents cross-contamination of the samples 104 as well as preventing another person administering the test from being potentially exposed to the agent (if present). The cap 112 is placed on the container 110 to seal the container 110. The container 110 may be marked with identifying information if desired along with the date and time, as well as any other pertinent information, prior to delivering the container 110 to a lab for testing. The ease of this process lends it to home use as well as in the workplace, and so forth.

Since the mask 102 is not destroyed, a fresh insert 902 may then be coupled to the mask 102, allowing the user to continue using the face covering uninterrupted. Inserts 902 may be comprised of various paper and/or cloth combinations for performance and economy. Inserts 902 may also be formed using other natural and man-made materials.

In various implementations, additional or alternative components, techniques, sequences, or processes may be used to implement the techniques described herein. The components and/or techniques may be arranged and/or combined in various combinations, while resulting in similar or approximately identical results. It is to be understood that the techniques described may be implemented in a stand-alone manner or as part of a larger system (e.g., integrated with other systems, techniques, protocols, etc.).

### EXAMPLE PILOT

In an initial pilot program, the process 1400 was utilized in several workplace facilities to effectively detect SARS-CoV-2 shed by both symptomatic and pre-symptomatic individuals. Testing up to 84 masks 102 per test day over the course of one year detected SARS-CoV-2 virus in 13 samples 104. Of these samples 104, eight came from masks 102 worn by asymptomatic/pre-symptomatic individuals who did not display fever >38° C. during standard temperature screening protocols. Individuals associated with the masks 102 on which SARS-CoV-2 was detected as well as other individuals in close contact were considered potentially exposed and advised to seek COVID-19 testing through medical providers. Diagnostic test results were available for 11 of these individuals, with 10 being confirmed positive for COVID-19.

Early identification of virus shed by pre-symptomatic individuals can lead to reduced viral contamination of the environment and decreased risk of ongoing transmission. During the period of the pilot program, the local per capita transmission rate reached over seven cases per 1000 residents. Despite this high local risk, the pilot facilities experienced no congregate outbreaks and had no indication of any missed infections, with over 5000 samples 104 tested in total.

### CONCLUSION

While various discreet embodiments have been described throughout, the individual features of the various embodiments may be combined to form other embodiments not specifically described. The embodiments formed by combining the features of described embodiments are also within the scope of the disclosure.

What is claimed is:

1. A method for detecting an infectious agent, comprising:
retrieving a face covering that contains one or more of oral secretions, nasal secretions, skin secretions, and skin flora on or within a material of the face covering;
removing one or more samples of the material of the face covering that contains the one or more of the oral secretions, nasal secretions, skin secretions, and skin flora from the face covering;
depositing the one or more samples into a predetermined formulation;
processing the one or more samples with the formulation; and
testing the one or more of the oral secretions, nasal secretions, skin secretions, and skin flora for the presence of an infectious agent.

2. The method of claim 1, further comprising wearing the face covering over a mouth and/or a nose for a predetermined period of time.

3. The method of claim 1, further comprising accumulating the one or more of the oral secretions, nasal secretions, skin secretions, and skin flora into the face covering while the face covering is worn over a mouth and/or a nose.

4. The method of claim 1, further comprising folding the face covering and cutting a plurality of the samples of the material of the face covering that contains the one or more of the oral secretions, nasal secretions, skin secretions, and skin flora from the face covering with a single cut, wherein a quantity of the samples of the plurality of samples is dependent on a number of folds of the face covering.

5. The method of claim 1, further comprising immersing the one or more samples of the material of the face covering that contains the one or more of the oral secretions, nasal secretions, skin secretions, and skin flora with the formulation and storing the immersed samples at room temperature for a predetermined duration.

6. The method of claim 5, wherein the predetermined duration is at least one month, and wherein nucleic acid of the infectious agent within the immersed samples is preserved and detectable over the at least one month.

7. The method of claim 5, further comprising inactivating enzymes within the one or more of the oral secretions, nasal secretions, skin secretions, and skin flora while preserving genome components of the infectious agent within the one or more of the oral secretions, nasal secretions, skin secretions, and skin flora with the formulation.

8. The method of claim 1, further comprising eluting biological matter within the one or more of the oral secretions, nasal secretions, skin secretions, and skin flora from the one or more samples of the material of the face covering that contains the one or more of the oral secretions, nasal secretions, skin secretions, and skin flora without dissolving the material of the face covering.

9. The method of claim 8, further comprising separating the infectious agent from a remainder of the biological matter via the formulation.

10. The method of claim 1, further comprising isolating the infectious agent from the material of the face covering of the one or more samples via the formulation and identifying a concentration or population of the infectious agent.

11. The method of claim 1, wherein the face covering comprises a clinical face mask.

12. The method of claim 1, wherein the face covering is comprised of one or more of paper, textile, polymer, a synthetic material, or a naturally occurring material.

13. The method of claim 1, wherein the face covering comprises an insert removably coupled to a face mask.

14. A method for detecting an infectious agent, comprising:
retrieving a face covering worn over the mouth and/or nose of a person, wherein the face covering contains one or more of oral secretions, nasal secretions, skin secretions, and skin flora deposited on or into material of the face covering by the person while worn;
cutting a sample from a central region of the face covering that contains the one or more of the oral secretions, nasal secretions, skin secretions, and skin flora;
depositing the sample into a collection container that includes a predetermined formulation;
immersing the sample with the formulation;
separating the one or more of the oral secretions, nasal secretions, skin secretions, and skin flora from the material of the face covering of the sample with the formulation; and
testing the one or more of the oral secretions, nasal secretions, skin secretions, and skin flora for the presence of an infectious agent.

15. The method of claim 14, further comprising folding the face covering one or more times and cutting multiple samples from the folded face covering using a single cut from a hole punch tool.

16. The method of claim 14, further comprising inactivating enzymes within the one or more of the oral secretions, nasal secretions, skin secretions, and skin flora and preserving genetic material of the infectious agent if present in the one or more of the oral secretions, nasal secretions, skin secretions, and skin flora.

17. The method of claim 16, wherein the preserving comprises preserving the genetic material at room temperature for over one month.

18. The method of claim 14, wherein the formulation comprises a solution of a chaotrope, alcohol, and ionic detergent.

19. A system comprising:
a face covering configured to be worn over a mouth and/or a nose and to collect one or more of oral secretions, nasal secretions, skin secretions, and skin flora on or within a material of the face covering;
a cutting device configured to remove a sample of the material of the face covering that contains the one or more of the oral secretions, nasal secretions, skin secretions, and skin flora from the face covering;
a sealable storage container configured isolate the sample from the environment and to store or transport the sample; and
a formulation within the container configured to cover the samples or to be absorbed by the material of the samples and to preserve biological matter within the one or more of the oral secretions, nasal secretions, skin secretions, and skin flora at room temperature for at least one month based on the composition of the formulation.

20. The system of claim 19, further comprising a tool for handling or removing the cut sample from the face covering.

21. The system of claim 19, wherein the cutting device is configured to cut multiple layers of the face covering when the face covering is folded.

22. The system of claim 19, wherein the formulation is configured to inactivate enzymes within the one or more of the oral secretions, nasal secretions, skin secretions, and skin flora while preserving genome components of the infectious agent within the one or more of the oral secretions, nasal secretions, skin secretions, and skin flora.

23. The method of claim 19, wherein the face covering comprises an insert removably coupled to a face mask.
